(19) United States
(12) Patent Application Publication
(10) Pub. No.: US 2023/0330060 A1
(43) Pub. Date: Oct. 19, 2023

(54) COMPOUNDS FOR TREATMENT OF HEART FAILURE

(71) Applicant: Sulfateq B.V., Groningen (NL)

(72) Inventors: Robert Henk HENNING, Groningen (NL); Adrianus Cornelis VAN DER GRAAF, Groningen (NL); Guido KRENNING, Groningen (NL); Lucas Moritz WIGGENHAUSER, Tübingen (DE)

(21) Appl. No.: 18/026,834

(22) PCT Filed: Sep. 21, 2021
(86) PCT No.: PCT/EP2021/075967

§ 371 Date: Mar. 17, 2023

(30) Foreign Application Priority Data
Sep. 21, 2020 (NL) 2026511

(51) Int. Cl. A61K 31/353; A61K 31/453; A61K 31/496; A61P 9/04

(52) U.S. Cl. CPC A61K 31/353; A61K 31/453; A61K 31/496; A61P 9/04

(57) ABSTRACT

The invention relates to certain chromanol, quinone or hydroquinone compounds and derivatives thereof for treatment of heart failure with reduced ejection fraction (HFrEF). Specifically, the present invention relates to chromanol compounds chosen from S-(6-hydroxy-2,5,7,8-tetramethylchroman-2yl)(piperazin-1-yl)methanone and S-(6-hydroxy-2,5,7,8-tetramethylchroman-2-yl)(4-(2-hydroxyethyl)piperazin-1-yl)methanone, and pharmaceutically acceptable salts thereof.

### I. FIELD OF THE INVENTION

The invention relates to chromanol compounds and derivatives thereof for treatment or prophylaxis of heart failure with reduced ejection fraction (HFrEF).

### II. DESCRIPTION OF THE BACKGROUND ART

Heart failure is a clinical diagnosis, characterized by symptoms and signs including breathlessness, fatigue, and elevated venous pressure, caused by an evidenced abnormal cardiac function (Pearse and Cowie 2014).

There are different types of heart failure, which generally are be classified as heart failure with reduced ejection fraction (HFrEF), heart failure with preserved ejection fraction (HFpEF), and congestive heart failure. Generally, these conditions are treated differently, and medicaments suitable for one type of heart failure are generally not suitable to treat other types of heart failure.

HFrEF is quantified with reference to the left ventricular ejection fraction (LVEF), usually derived from echocardiography, with values of more than 50-60% accepted as normal. Patients suffering from heart failure with preserved ejection fraction also demonstrate values ≥50%. Values below 40% are considered a reduced LVEF (HFrEF) and patients with an LVEF in the range of 40-49% represent a ‘grey area’, which is defined as heart failure with a mildly reduced ejection fraction (HFmrEF). Patients with HFmrEF most probably have primarily mild systolic dysfunction, but also with features of diastolic dysfunction.

Differentiation of patients with HF based on LVEF is important due to different underlying aetiologies, demographics, co-morbidities and response to therapies.

Asymptomatic structural or functional cardiac abnormalities (for example systolic or diastolic left ventricular (LV) dysfunction), are precursors of heart failure. However, abnormalities of the valves, pericardium, endocardium, heart rhythm and conduction can also cause heart failure (and more than one abnormality is often present).

Patients suffering from heart failure with reduced ejection fraction have a left ventricular systolic dysfunction commonly combined with elements of diastolic dysfunction as well. (Yancy et al. JACC Vol 62, No. 16. 2013)

The identification of the underlying cardiac problem of heart failure is crucial for therapeutic reasons, as the precise pathology determines the specific treatment used (e.g. valve repair or replacement for valvular disease, specific pharmacological therapy for HF with reduced EF, reduction of heart rate in tachycardiomyopathy, etc). (ESC Guidelines 2016).

WO2020/096862 describes a cardiac device for remodelling a ventricle, the device comprising a means for distributing force configured to extend from a first ventricle wall to a second ventricle wall and a first plurality of means for anchoring configured to secure the means for distributing force to a first area of tissue at the first ventricle wall as a percutaneous treatment of heart failure with reduced ejection fraction.

RU2422136 discloses a method of treating chronic heart failure with reduced left ventricular ejection fraction using a β-blocker, a diuretic a recombinant human interleukin and an ACE inhibitor.

Despite the availabilities of such methods, there remains a need for new methods or compounds for the treatment of heart failure with reduced ejection fraction.

It is an object of the present invention to provide compounds for the treatment or prophylaxis of heart failure with reduced ejection fraction (HFmrEF or HFrEF).

### III. BRIEF SUMMARY OF THE INVENTION

The above object is met by providing certain chromanol, quinone or hydroquinone compounds for use in such treatment.

The above object is met by the present invention by providing compounds according to formula (I), (II), the hydroquinone analogue of formula (II), or a pharmaceutically acceptable salt thereof, for use in the treatment or prophylaxis of heart failure with reduced ejection fraction;

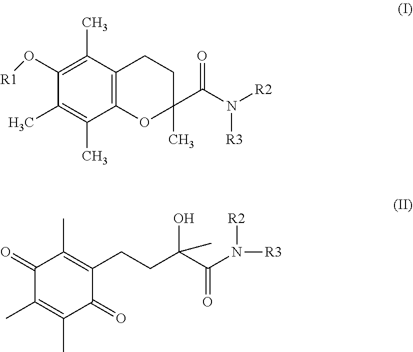

wherein R1 represents a hydrogen or prodrug moiety that can be removed in living tissue
and wherein either
   R2 and R3 together with the N atom to which they are attached form a saturated or unsaturated, non-aromatic, optionally substituted, 5-8 membered ring, having one to four N, O, or S atoms, wherein R2 and R3 together contain 3-12 carbon atoms;
   or R2 is a hydrogen atom, or an alkyl group with 1-6 carbon atoms, and R3 is an alkyl group, optionally substituted with nitrogen or oxygen, wherein the alkyl group comprises 3-12 carbon atoms, the alkyl group in R3 comprises one or more non-aromatic cyclic structures that may comprise nitrogen or oxygen atoms in the ring, and may contain linear and/or branched substituted groups, and one or more ethylenic unsaturations.

For the present invention, the compound according to formula (II) includes the hydrogenated quinone (i.e. the hydroquinone) analogue, although the quinone derivative is preferred in view of stability.

The compound of Formula II is one of the metabolites of a compound according to Formula I. Thus, the compound of Formula I is a pro-drug for the compound of Formula II, wherein both

In a preferred embodiment, the nitrogen can be amine, quaternary amine, guanidine or imine and oxygen is hydroxyl, carbonyl or carboxylic acid; and/or oxygen and nitrogen together may form amide, urea or carbamate groups.

In a preferred embodiment, R1 in formula (I) is hydrogen or forms together with the 6-oxygen an ester group with 2-6 carbon atoms.

In a preferred embodiment of either compounds according to formula (I) or according to formula (II), R2 and R3 together with the N atom to which they are attached form a saturated ring incorporating an additional N atom, which ring is unsubstituted or substituted with an alcohol, or alkanol group having 1-4 carbon atoms, such as ethylol.

In another preferred embodiment R2 is a hydrogen atom and R3 comprises a saturated cyclic structure having 4-7 carbon atoms and having one nitrogen atom, which ring may be substituted with an alkyl group, alcohol group, or with a group with 1-4 carbon atoms that may comprise an oxygen, carboxylic acid or amine group.

In another preferred embodiment the compound is a compound according to formula II and R2 is a hydrogen atom and R3 comprises a cyclic structure having 4-6 carbon atoms and having one nitrogen atom which ring is unsubstituted or substituted with an alcohol, or alkanol group having 1-4 carbon atoms, such as ethylol, and preferably is optionally substituted with methyl, ethyl, or alcohol substituted methyl or ethyl.

In another preferred embodiment, the compound is a compound according to formula I, R2 is a hydrogen atom and R3 comprises a saturated cyclic structure having 4-7 carbon atoms and having one nitrogen atom, which ring is unsubstituted or substituted with an alcohol, or alkanol group having 1-4 carbon atoms, such as ethylol, and preferably is optionally substituted with methyl, ethyl, or alcohol substituted methyl or ethyl.

According to yet another preferred embodiment, the compound is either (6-hydroxy-2,5,7,8-tetramethylchroman-2yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13), or (6-hydroxy-2,5,7,8-tetramethylchroman-2-yl)(4-(2-hydroxyethyl)piperazin-1-yl)methanone (SUL-109) or a pharmaceutically acceptable salt thereof, as racemic mixture or as one of its enantiomers.

Although the S enantiomers are effective, in a most preferred embodiment, the compound is the (2R)-enantiomer of SUL-121, namely (2R)-(6-hydroxy-2,5,7,8-tetramethylchroman-2yl)(piperazin-1-yl)methanone (SUL-150) or a pharmaceutically acceptable salt thereof, as the R enantiomer appears even more effective.

In a preferred embodiment according to the invention, the compound either according to formula (I) or according to formula (II) has a molecular weight lower than 500 Da.

In a preferred embodiment according to the invention, the compound either according to formula (I) or according to formula (II) is for use for treating or prophylaxis of heart failure with reduced ejection fraction (HFmrEF) wherein the ejection fraction is reduced to 50% or less, and even more preferred, the compound either according to formula (I) or according to formula (II) is for use for treating or prophylaxis of heart failure with reduced ejection fraction (HFrEF) wherein the ejection fraction is reduced to 40% or less.

### IV. SHORT DESCRIPTION OF THE FIGURES

FIG. 1 shows that SUL-150 administration precludes at least to a certain extent the development of heart failure with reduced ejection fraction. Chronic doxorubicin administration in rats results in heart failure with reduced ejection fraction, characterized by a decrease in (A) heart rate, (B) ejection fraction, (C) stroke work and (D) cardiac output. The oral administration of SUL-150, either prophylactically or therapeutically, precludes at least part of these changes in cardiac function. *p<0.05 versus sham, †p<0.05 versus doxorubicin/vehicle.

FIG. 2 shows that EGFP synthesis associates with H9C2 cardiomyocyte cell size and protein synthesis. (A) Phenylephrine dose-dependently increases H9C2 cardiomyocyte cell surface area and (B) EGFP expression. (C) H9C2 cardiomyocyte cell surface area is associated to EGFP expression. (D) Phenylephrine dose-dependently increases protein synthesis of H9C2 cardiomyocytes. (E) H9C2 cardiomyocyte protein synthesis is associated with the expression of EGFP. (F) Phenylephrine induces EGFP expression in H9C2 cardiomyocytes as compared to vehicle-treated control cardiomyocytes. The inhibitor of protein synthesis Brefeldin A reduces the expression of EGFP in H9C2 cardiomyocytes

FIG. 3 shows that SUL compounds inhibit phenylephrine-induced hypertrophic responses in rat H9C2 cardiomyocytes. Phenylephrine dose-dependently induces the expression of EGFP in H9C2 cardiomyocytes (grey line, figs A-H). The preincubation with 30 μM (A) SUL-11, (B) SUL-99, (C) SUL-127, (D) SUL-13, (E) SUL-138 or (F) SUL-138′s primary metabolite SUL-138M2, (G) SUL-150 or (H) SUL-151 reduced the expression of EGFP by cardiomyocytes

FIG. 4. shows that SUL-150 administration precludes doxorubicin-induced cardiac fibrogenesis. Chronic doxorubicin administration in rats results in fibrogenesis, characterized by an increase in collagen deposition in between cardiomyocytes. The oral administration of SUL-150, either prophylactically or therapeutically, precludes cardiac fibrogenesis resulting in a lower collagen content in the cardiac tissue.

FIG. 5. shows that SUL-150 administration mitigates cardiac oxidative stress. Chronic doxorubicin administration induces cardiac oxidative stress as indicated by increased lipid peroxidation products (TBARS, A). Oral SUL-150 administration, either in a prophylactic or therapeutic regime precludes the induction of cardiac oxidative stress and maintains the level of lipid peroxidation products at baseline (A). As oxidative stress may arise from an increased radical production or a decreased radical scavenging activity, cardiac radical scavenging was investigated (B). Neither doxorubicin administration, nor SUL-150 administration altered the cardiac radical scavenging activity.

FIG. 6. shows that SUL-150 administration maintains cardiac energy levels and mitochondrial copy number. Chronic doxorubicin administration depletes cardiac ATP levels (normalized for ADP), suggestive of mitochondrial dysfunction (A). SUL-150 administration, either prophylactically or in a therapeutic regime mitigates the cardiac energy loss. Chronic doxorubicin administration reduces the cardiac mtDNA copy number (B), which may underly the loss of ATP. SUL-150 mitigates the reduction in mtDNA copy numbers. In doxorubicin-treated rats, mtDNA copy number associated with cardiac ejection fraction (EF), suggesting that the maintenance of cardiac mitochondrial mass underlies the improved cardiac contractility (C).

FIG. 7. shows that SUL-150 maintains the activity of respiratory complex IV under doxorubicin stress. H9C2 cardiomyoblasts were exposed to doxorubicin (1 μM) for 24 hours, after which their mitochondria were isolated and assessed for complex IV activity.

Doxorubicin exposure reduced complex IV activity, which is mitigated by co-treatment of H9C2 cardiomyoblasts with SUL-150.

### V. DETAILED DESCRIPTION OF THE INVENTION

The object of the present invention, to provide compounds for the treatment or prophylaxis of heart failure with reduced ejection fraction (HFrEF) is met by providing compounds according to formula (I) or (II), as shown above, or a pharmaceutically acceptable salt thereof for use in the treatment or prophylaxis of HFrEF.

The treatment or prophylaxis with the chromanol, quinone or hydroquinone compounds according to the present invention preferably is part of a combination therapy with one or more common other measures to treat heart failure.

R1 can be a substituent that is easily removed in the human body, such that the compound is a prodrug. R1 can be for example an amino acid derivative or ester derivative, and generally has a molecular weight lower than 100 dalton.

In a preferred embodiment, R1 in formula (I) is hydrogen or forms together with the 6-oxygen an ester group with 2-6 carbon atoms. The ester can comprise one or more ether or alcohol groups. Suitable esters are acetate, butyrate, 3-hydroxy butyrate and the like.

In a preferred embodiment of either compounds according to formula (I) or according to formula (II), R2 and R3 together with the N atom to which they are attached form a saturated ring having 3-6 carbon atoms and incorporating one additional N atom, which may be substituted with 1-4 carbon atoms that may comprise an oxygen, carboxylic acid or amine group.

More preferably, R2 and R3 together with the N atom to which they are attached form a 5-7 membered ring comprising one additional amine group, which ring is optionally substituted with methyl, ethyl, or alcohol substituted methyl or ethyl.

In another preferred embodiment, R2 is a hydrogen atom and R3 comprises a cyclic structure having 3-6 carbon atoms and having one nitrogen atom.

More preferably, R2 is a hydrogen atom, and R3 comprises a 5-7 membered ring comprising one additional amine group, which ring is attached to the amide-nitrogen, and which ring is optionally substituted with methyl, ethyl, or alcohol substituted methyl or ethyl.

In either case, the ring (the cyclic structure formed by R2 and R3, or of R3 alone) may be unsubstituted or substituted with an alkyl having 1-4 carbon atoms, alcohol, or alkanol group having 1-4 carbon atoms, such as ethylol.

In a preferred embodiment according to the invention, the compound either according to formula (I) or according to formula (II) has a molecular weight lower than 500 Da.

In a preferred embodiment, the compound for use according the present invention is a chromanol compound according to formula I.

Certain chromanol compounds have been described in WO2014/098586. The compounds described in detail have abbreviations, referring to SUL-XXX (XXX being a 2 or 3 digit number). Many of these compounds are racemic mixtures, although some enantiomers have been tested as well. Suitable methods to prepare chromanol compounds according to the present invention are described in WO2014/098586 or WO2014/011047.

WO 2017/060432 A1 discloses amide-derivatives of 2-hydroxy-2-methyl-4-(3,5,6-trimethyl-1,4-benzoquinon-2-yl)-butanoic acid and methods of making such compounds.

Hydrogenated quinone derivatives can be easily prepared by hydrogenation of the quinone structure.

According to yet another preferred embodiment, the compound is either (6-hydroxy-2,5,7,8-tetramethylchroman-2yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13), or (6-hydroxy-2,5,7,8-tetramethylchroman-2-yl)(4-(2-hydroxyethyl)piperazin-1-yl)methanone (SUL-109) or a pharmaceutically acceptable salt thereof, as racemic mixture or as one of its enantiomers.

In a most preferred embodiment, the compound is the R-enantiomer of SUL-121, namely R-(6-hydroxy-2,5,7,8-tetramethylchroman-2yl)(piperazin-1-yl)methanone (SUL-150) or a pharmaceutically acceptable salt thereof.

The counterion in the pharmaceutically acceptable salt can be a counterion as known in the art. Preferably, the compounds have at least one basic nitrogen, an amine, which can be protonated. The counterion preferably is a halogen such as chloride, sulphate, citrate, formate or the like, and most preferably chloride.

The compounds are effective as a racemic mixture or in a substantially pure enantiomeric form. The compounds have one or more chiral centers, generally one or two.

Preferably, the compound is a substantially enantiomerically pure compound. Substantially enantiomerically pure is about 95% enantiomeric excess or more, more preferably about 98% enantiomeric excess, and most preferably about 99% or more enantiomeric excess. Also, in case the compound contains more than one chiral center, these amounts apply.

The compounds are preferably used in effective amounts, to achieve treatment or prophylaxis of HFrEF. HFrEF wherein the ejection fraction is about 40% or less.

The wording treatment or prophylaxis includes amelioration of the symptoms of heart failure and/or reduction in progress of heart failure, including improvement of heart function such as heart rate and cardiac output.

Preferably, the compounds according to the invention are for use of treatment or prophylaxis of HFrEF in organs in mammals, wherein the mammal is preferably human.

HFrEF is quantified with reference to the left ventricular ejection fraction (LVEF), usually derived from echocardiography, with values of more than 50-60% accepted as normal. Patients suffering from heart failure with preserved ejection fraction also demonstrate values ≥50%. Values below 40% are considered a reduced LVEF (HFrEF) and patients with an LVEF in the range of 40-49% represent a ‘grey area’, which is defined as heart failure with a mildly reduced ejection fraction (HFmrEF). Patients with HFmrEF most probably have primarily mild systolic dysfunction, but also with features of diastolic dysfunction.

The present invention provides compounds for use in a treatment of HFrEF with a preserved ejection fraction of 50% or lower (HFmrEF) and of about 40% or lower (HFrEF). Preferably, the compounds for use in a treatment are for treating HFrEF of about 40% or lower preserved ejection fraction.

Effects generally are observed with amounts of about 1 μM in body fluid, but preferably higher amounts are used. Preferred amounts are concentrations in vivo or in vitro of about 10 μM or higher, more preferably about 20 μM or higher. Generally, a concentration in human of about 200 μM or lower should be sufficient and safe.

For human use, this would mean—assuming a 30 L distribution volume, 100% availability and a concentration of about 1 μM—a dosage of about 10 mg or more. Preferred amounts would result in a concentration of about 10 μM — for which a dosage of about 100 mg or more would be suitable. Hence, preferably, dosage forms of about 20 mg or more, preferably 50 mg or more, preferably 100 mg or more are suitable.

Generally, solid, oral dosage forms contain as a maximum about 500 mg compound, preferably about 450 mg or less, to allow for excipients.

With parenteral administration, such as for example i.v., or with other liquid forms of administration, larger amounts can be administered.

Examples of dosages which can be used are an effective amount of the compounds of the invention of a dosage of 0.2 mg/kg or higher, such as preferably within the range of about 1 mg/kg to about 100 mg/kg, or within about 2 mg/kg to about 40 mg/kg body weight, or within about 3 mg/kg to about 30 mg/kg body weight, or within about 4 mg/kg to about 15 mg/kg body weight. Compounds of the present invention may be administered in a single daily dose, or the total daily dosage may be administered in divided dosage of two, three or four times daily.

The compounds described herein can be formulated as pharmaceutical compositions by formulation with additives such as pharmaceutically or physiologically acceptable excipients carriers, and vehicles.

Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, cyclodextran, starch, gelatin, cellulose, methyl cellulose, sodium carboxymethyl cellulose, dextrose, hydroxypropyl-P-cyclodextrin, polyvinylpyrrolidone, low melting waxes, and the like, as well as combinations of any two or more thereof. Other suitable pharmaceutically acceptable excipients are described in “Remington's Pharmaceutical Sciences,” Mack Pub. Co., New Jersey (1991).

A pharmaceutical composition preferably comprises a unit dose formulation, where the unit dose is a dose sufficient to have a therapeutic effect. The unit dose may be a dose administered periodically in a course of treatment or suppression of a disorder.

The compounds of the invention may be administered enterally, orally, parenterally, sublingually, by inhalation (e. g. as mists or sprays), rectally, or topically in dosage unit formulations containing conventional nontoxic pharmaceutically or physiologically acceptable carriers, adjuvants, and vehicles as desired. The term parenteral as used herein includes subcutaneous injections, intravenous, intramuscular, intratarsal injection, or infusion techniques. The compounds are mixed with pharmaceutically acceptable carriers, adjuvants, and vehicles appropriate for the desired route of administration.

Generally, oral administration is a preferred route of administration, and formulations suitable for oral administration are preferred formulations.

The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at room temperature but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavouring, and perfuming agents.

The amount of active ingredient that may be combined with the carrier materials to produce a single dosage form will vary depending upon the host to which the active ingredient is administered and the particular mode of administration. The unit dosage chosen is usually fabricated and administered to provide a defined final concentration of drug in the blood, tissues, organs, or other targeted region of the body. The effective amount for a given situation can be readily determined by routine experimentation and is within the skill and judgment of the ordinary clinician or skilled person.

The present invention will be further illustrated using the examples below. In the examples, reference is made to figures.

### VI. EXAMPLES

### Example 1

The effectiveness of the compounds according to the invention for treatment or prophylaxis of HFrEF was tested in vivo in rats. Doxorubicin, an anthracycline antibiotic used in cancer chemotherapy, was employed to induce heart failure with reduced ejection fraction in preclinical rat models (Christiansen et al. (2006) Eur. J. Cardiothorac. Surg. 30:611-616; Ertunc et al. (2009) Pharmacology 84:240-248; Hayward et al. (2007) J. Am. Ass. Lab. Animal Sci. 46:20-32).

Experimental

SUL-150 (250 mg) was dissolved in 100% EtOH (1.02 ml) and further diluted in distilled water (68.7 ml) to obtain a clear 10 mM solution. The SUL-150 solution was sprayed homogeneously over standard food pellets (1.25 kg). Food pellets were air-dried overnight. Food pellets were freshly prepared weekly. With the approximation of 200 mg/kg SUL-150 in the food pellets and a daily dietary intake of 25 gram for a 350 gram rat, daily dosing of SUL-150 approximates 5 mg per day.

Male outbred Wistar rats of 250-300 grams (10-12 weeks old) were kept on a 12h light/dark cycle and received a standard diet with distilled water ad libitum. Thirty rats were administered with doxorubicin (2 mg/kg) weekly via intraperitoneal injection for 9 consecutive weeks and were left either untreated (standard chow diet, 10 rats), or received a diet supplemented with SUL-150 as prophylaxis (prior to the 1st doxorubicin administration, 10 rats) or therapy (starting after the 6th doxorubicin administration, 10 rats). Sham control rats (8 rats) were administered with saline weekly via intraperitoneal injection for 9 consecutive weeks and received a standard diet. All rats were subjected to cardiac function assessment 12 weeks after the first doxorubicin administration.

Cardiac function tests (PV-Loop assessment) were performed under anaesthesia (isoflurane in O2 (FiO2 100%)) followed by orotracheal intubation. The rats were placed in a mechanical rodent ventilator (Harvard Apparatus, Holliston, Massachusetts) with tidal volume of 10 mL/kg and 70 breaths per minute. A small window was opened below the sternum and with a small incision a 2F microtip pressure-conductance catheter (SPR-838; Millar Instruments, Houston, Tx) was inserted directly on the left ventricle (LV) through the apex. After stabilization, signals were recorded with a pressure-volume conductance System (MPVSSUL-Ultra, Millar Instruments, Houston, Tx) connected to a data acquisition system (PowerLab, AD Instruments, Colorado Springs, Co).

Mean arterial pressure (MAP), LV stroke work (LVSW), stroke volume (SV), LV end-diastolic volume (LVEDV), LV end-systolic pressure (LVESP), ratio between mean arterial pressure and LV end-systolic pressure (MAP-LVESP), LV ejection fraction (LVEF), heart rate (HR), maximal slope of the LV systolic pressure increment (dP/dt max), and time constant of LV pressure decay (tau) were obtained at steady state conditions.

For the preload maneuver the inferior vena cava was compressed and data collected to assess the preload recruitable stroke work (PRSW), dP/dt-end-diastolic volume relation (dP/dt-EDV), slope of end-systolic and end-diastolic P-V relations (ESPVR and EDPVR).

Volume calibration was performed with fresh heparinized warm blood from each animal. Also, 50 mL of 7.5% hypertonic saline was injected at the end of each experiment for the parallel conductance volume calibration.

Data is analysed in GraphPad Prism 8.0 (GraphPad Software Inc, Ca). All results are expressed as mean±S.D. (standard deviation) or median and interquartile interval. The difference between groups was assessed by ANOVA followed by pairwise comparisons to the sham and vehicle treatment groups with p-values adjusted for multiple comparisons using FDR correction.

### RESULTS

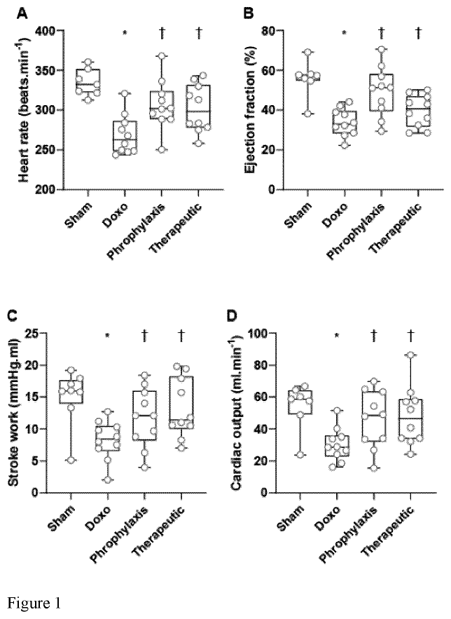

FIG. 1 shows that chronic doxorubicin administration in rats results in the development of heart failure as indicated by a reduction in heart rate, ejection fraction, and stroke work, culminating in a severe reduction in cardiac output (the * indicates p<0.05 with respect to sham). FIG. 1 further demonstrates that administration of SUL-150 via food pellets prior to the administration of doxorubicin (prophylaxis group), substantially preserves cardiac function and largely maintains ejection fraction and cardiac output (the † indicates p<0.05 with respect to the doxorubicin treated group). The administration of SUL-150 via food pellets after 6 cumulative doxorubicin administrations (therapeutic group) substantially improves the cardiac function in comparison to the non-treated group (p<0.05 in comparison to the doxorubicin treated group), and the parameters were not different from these as obtained for the prophylaxis group, demonstrating that SUL-150 is a therapeutic for heart failure.

Table 1 shows that doxorubicin-induced heart failure appears as heart failure with reduced ejection fraction (HFrEF) and is characterized by a decline in both contractile and relaxation capacity, indicative of remodelling or fibrogenesis within the cardiac tissue.

The administration of SUL-150, either in prophylaxis or therapeutic regime, maintains the contractile capacity (i.e. dP/dTmax, contractility efficiency) and relaxation capacity (i.e. Tau), suggesting, without wishing to be bound by theory, that SUL-150 either inhibits cardiac fibrogenesis or increases contractile energy.

### CONCLUSIONS

Doxorubicin-induced heart failure is characterized by a reduction in cardiac contraction and thus cardiac output. Treatment with the 6-chromanol SUL-150 substantially improves cardiac contractile function and thus cardiac output.

TABLE 1
Cardiac function assessment by PV-loop analysis

| | Doxorubicin | | | |
|---|---|---|---|---|
| | | | SUL-150 | |
| Parameter | Sham | Vehicle | Prophylaxis | Therapeutic |

Left Ventricular Pressure-Volume Measurements

| | | | | |
|---|---|---|---|---|
| ESP (mmHg) | 115.3 ± 15.4 | 107.0 ± 28.9 | 104.4 ± 12.3 | 114.3 ± 18.4 |
| EDP (mmHg) | 8.6 ± 2.0 | 7.1 ± 3.3 | 8.1 ± 3.0 | 7.4 ± 3.7 |
| ESV (μl) | 151.4 ± 79.1 | 222.8 ± 79.3 | 175.3 ± 107 | 205.0 ± 96.4 |
| EDV (μl) | 313.0 ± 113 | 333.8 ± 99.2 | 325.0 ± 144 | 363.1 ± 115 |
| SV (μl) | 161.6 ± 41.5 | 111.0 ± 30.* | 149.7 ± 55.† | 158.1 ± 55.† |

Contractile parameters

| | | | | |
|---|---|---|---|---|
| dP/dTmax | 7227 ± 827 | 5126 ± 1519* | 6144 ± 1782 | 6487 ± 1665† |
| (mmHg · s−1) | | | | |
| Contractility | 68.8 ± 17.8 | 29.7 ± 11.4* | 53.0 ± 16.4† | 47.1 ± 18.1† |
| efficiency (%) | | | | |
| Ees (ESPVR) | 1.3 ± 0.7 | 0.6 ± 0.3* | 0.7 ± 0.6 | 0.5 ± 0.4 |
| (mmHg · μl−1) | | | | |
| PRSW | 14.1 ± 6.2 | 8.5 ± 2.1* | 9.9 ± 4.0 | 11.0 ± 3.9 |
| (mmHg · ml−1) | | | | |
| Ea (mmHg · μl−1) | 0.8 ± 0.3 | 1.0 ± 0.3* | 0.7 ± 0.2† | 0.7 ± 0.2† |

Relaxation parameters

| | | | | |
|---|---|---|---|---|
| dP/dTmin | −6034 ± 1136 | −4268 ± 1570* | −5006 ± 1140 | −5092 ± 1447 |
| (mmHg · s−1) | | | | |
| Emax (EDPVR) | 0.03 ± 0.01 | 0.03 ± 0.02 | 0.04 ± 0.03 | 0.03 ± 0.02 |
| (mmHg · μl−1) | | | | |
| Tau | 9.2 ± 1.2 | 17.2 ± 7.3* | 12.5 ± 3.1† | 13.6 ± 5.2† |

ESP = End-systolic pressure, EDP—end-diastolic pressure, ESV = end-systolic volume, EDV = end-diastolic volume, SV = stroke volume, Ees (ESPVR) = end-systolic elastance (slope of the end-systolic pressure volume relation), PRSW = preload recruitable stroke work, Ea = Arterial elastance, Emax (EDPVR) = maximal elastance (slope of the end-diastolic pressure volume relationship.
*p < 0.05 versus sham;
†p < 0.05 versus vehicle.

### Example 2

Example 2 shows in an in vitro experiment, that several different compounds according to the invention have improved efficacy in comparison to Trolox and some other Trolox derivatives that are not according the invention.

Ventricular hypertrophy is an ominous escalation of hemodynamically stressful conditions such as hypertension and valve disease which is directly related to heart failure with a reduced ejection fraction (HFrEF).

Ventricular hypertrophy in heart failure is attributable to an increase in heart mass and asymmetric interventricular septal thickening. Histological hallmarks include an increase in cardiomyocyte size (i.e. cardiomyocyte hypertrophy), disorganized arrangement of myocytes (myofibrillar disarray), and both perivascular and interstitial fibrosis, a common phenotype in many cardiomyopathies.

A characteristic feature of pathological cardiomyocyte hypertrophy is a switch in the gene expression profile, including the upregulation of the fetal cardiac myosin heavy chain-beta (MHC-β) in lieu of the adult predominant alpha isoform (MHC-α), skeletal alpha actin (SKA), and atrial natriuretic factor (ANF) genes. Additionally, cardiomyocytes switch to carbohydrate-dependent energetic machinery instead of fatty acid oxidation, which in turn necessitates alterations in expression levels of metabolic genes. Interestingly, both pathophysiological adaptations are associated with increased transcriptional activity downstream of CREB, JNK, NFκB and NFAT.

In vitro assays to investigate cardiomyocyte hypertrophy and screen for putative small molecule inhibitors of cardiomyocyte hypertrophy utilize well-characterized inducers of hypertrophy (e.g. phenylephrine or IL-6) combined with transcriptomic screens of typical genes upregulated during cardiomyocyte hypertrophy and the quantification of cardiomyocyte cell surface area. However, these assays are laborious and time-intensive and non-permissive for the rapid screening of a larger set of small molecules.

An alternative method that indirectly quantifies protein synthesis and cardiomyocyte cell size is the quantification of the expression of enhanced green fluorescent protein (EGFP) under the control of a standard cytomegalovirus (CMV) enhancer/promoter element [Vettel, 2012]. The CMV promoter contains multiple functional binding sites for CREB, NFκB and NFAT. Hence, the expression of EGFP by cardiomyocytes in vitro can be used as a high-throughput platform to screen for small molecules that inhibit cardiomyocyte hypertrophy [Vettel, 2012] and may have clinical efficacy in the treatment of heart failure.

The following compounds were tested (Table 2)

TABLE 2

| Compound | Chemical Name | Formula | Structure | MW |
|---|---|---|---|---|
| SUL-11 | 6-hydroxy-2,5,7,8- tetramethylchroman-2- carboxylic acid (Trolox) | C₁₄H₁₈O₄ | 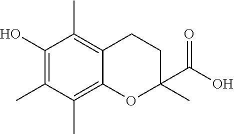 [SMILES: Cc1c(C)c2c(c(C)c1O)CCC(C)(C(=O)O)O2] | 250.3 |
| SUL-99 | methyl [(6-hydroxy- 2,5,7,8-tetramethyl-3,4- dihydro-2H-1- benzopyran-2- carbonyl)amino]acetate | C₁₇H₂₃NO₅ | 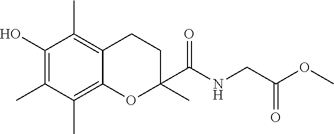 [SMILES: COC(=O)CNC(=O)C1(C)CCc2c(C)c(O)c(C)c(C)c2O1] | 321.4 |
| SUL-127 | methyl 6-hydroxy- 2,5,7,8-tetramethyl-3,4- dihydro-2H-1- benzopyran-2- carboxylate | C₁₅H₂₀O₄ | 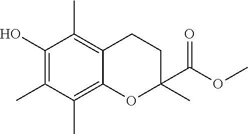 [SMILES: COC(=O)C1(C)CCc2c(C)c(O)c(C)c(C)c2O1] | 264.3 |
| SUL-13 | (S)-6-hydroxy-2,5,7,8- tetramethyl-N-((R)- piperidin-3- yl)chromane-2- carboxamide | C₁₉H₂₈N₂O₃ | 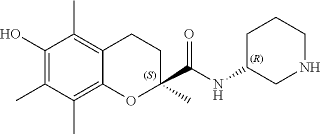 | 332.4 |
| SUL-138 | (S)-(6-hydroxy-2,5,7,8- tetramethylchroman-2- yl)(4-(2- hydroxyethyl)piperazin- 1-yl)methanone | C₂₀H₃₀N₂O₄ | 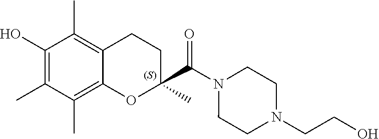 | 362.5 |
| SUL-138M2 | 4-(2,5-dihydroxy-3,4,6- trimethylphenyl)-2- hydroxy-1-(4-(2- hydroxyethyl)piperazin- 1-yl)-2-methylbutan-1- one | C₂₀H₃₂N₂ | 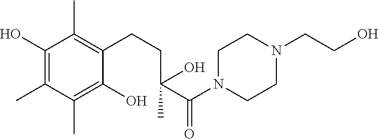 [SMILES: Cc1c(C)c(O)c(CC[C@@](C)(O)C(=O)N2CCN(CCO)CC2)c(C)c1O] | 380.5 |
| SUL-150 | (R)-(6-hydroxy-2,5,7,8- tetramethylchroman-2- yl)(piperazin-1- yl)methanone | C₁₈H₂₆N₂O₃ | 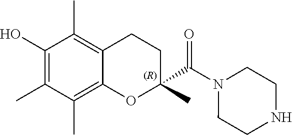 | 318.4 |
| SUL-151 | (S)-(6-hydroxy-2,5,7,8- tetramethylchroman-2- yl)(piperazin-1- yl)methanone | C₁₈H₂₆N₂O₃ | 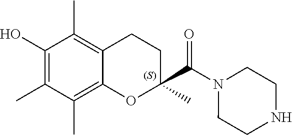 | 318.4 |

Experimental Design

H9C2 Cardiomyocyte Culture and Differentiation

Rat H9C2 cardiomyoblasts (ATCC CRL-1446) were maintained in DMEM medium containing 10% fetal bovine serum and 1% Penicillin-Streptomycin solution (Sigma-Aldrich) and passaged when the cultures reach a confluency of 70%. Prior to experiments, H9C2 myoblasts were differentiated to cardiomyocytes by serum reduction (to 1%) and stimulation with 20 nM retinoic acid for 5 days. Differentiated H9C2 cardiomyocytes were seeded at 0.6·10⁵ cells/cm² for all experiments.

Determination of Cell Size

H9C2 cardiomyocytes were serum starved for 24 h and thereafter exposed to different concentrations of phenylephrine (dose range 2·10⁻⁵ to 1·10⁻¹¹ M) for an additional 24 h. Cells were washed in ice-cold PBS and fixed by 2% paraformaldehyde in PBS at room temperature for 10 min. Fixed cells were incubated with 5 μM Rhodamine-conjugated Phalloidin (ThermoFisher Scientific) and washed extensively with PBS. Fluorescent pictures were taken randomly with an Zeiss AxioObserver Z1 microscope and cell size was analyzed with CellProfiler software [McQuin, 2018].

Quantification of EGFP Synthesis as Surrogate for Cell Size.

H9C2 cardiomyocytes (0.6·10⁵ cells/cm2) were infected with CMV-copGPF (MOI 10) in culture medium without serum for 24 h. H9C2 cardiomyocytes were pre-incubated with 30 μM SUL compounds under standard culture conditions for 30 min and then stimulated with phenylephrine (dose range 2·10⁻⁵ to 1·10⁻¹¹ M) for an additional period of 24 h. Thereafter H9C2 cardiomyocytes were lysed in 100 μl soft lysis buffer (25 mM Tris, 2 mM Dithiothreitol, 2 mM EDTA, 1% Triton X-100, pH 7.4). per well and fluorescence intensity of the obtained supernatant was recorded in a CLARIOStar Plus plate reader (BMG Labtech) equipped with a FITC filter set (excitation 488 nm with 10 nm bandwidth; emission 515 with a 20 nm bandwidth).

Statistical Evaluation

All experiments were performed in triplicate per condition and averaged. Data obtained from two individual experiments were used for evaluation in GraphPad Prism 8.0 (GraphPad Software Inc, Ca). Average H9C2 cardiomyocyte cell size was associated to average EGFP levels using linear regression. Phenylephrine-induced EGFP synthesis was normalized, using baseline levels as 0% and maximum EGFP recordings as 100%. All datasets were normalized to vehicle controls. 4 parameter non-linear regression was used to determine the efficacy of potency of phenylephrine to induce GFP synthesis. The efficacy of SUL compounds to inhibit phenylephrine-induced EGFP synthesis was calculated as 100%-Eₘₐₓ, wherein Emax is the maximal effect evoked by phenylephrine.

### RESULTS

EGFP Synthesis is Associated to H9C2 Cardiomyocyte Hypertrophy

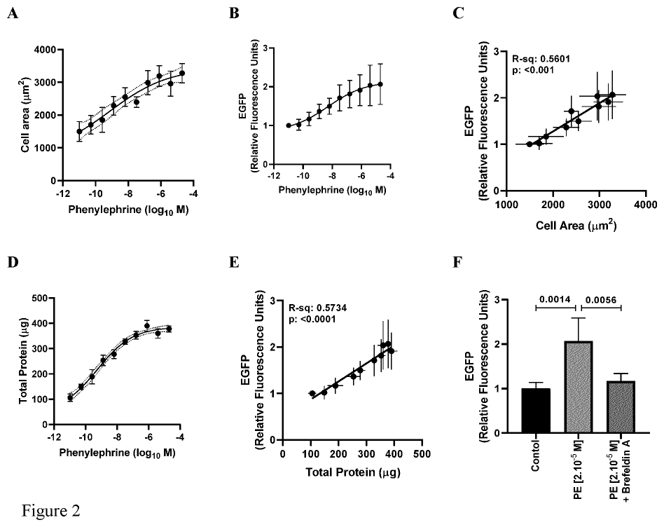

H9C2 cardiomyoblasts were differentiated into cardiomyocytes according to established protocols and stimulated with increasing doses of phenylephrine: (1·10⁻¹¹ to 2·10⁻⁵ M) to induce cardiomyocyte hypertrophy for 24 h. Phenylephrine dose-dependently (EC₅₀ is 7.4·10⁻¹⁰) increased H9C2 cardiomyocyte cell surface area from 791±263 μm² in vehicle-treated control cardiomyocytes to 3278±296 μm² in cardiomyocytes exposed to 2·10⁻⁵ M phenylephrine (FIG. 2A). Similarly, phenylephrine dose-dependently increased EGFP fluorescence (EC₅₀ is 5.2·10⁻⁹ M) in H9C2 cardiomyocytes transformed with CMV-EGFP lentiviral particles (FIG. 2B). The increase in H9C2 cardiomyocyte cell surface area associated to the expression of EGFP (R² is 0.5601, p<0.001; FIG. 2C). Similar to the increase in cell surface area, phenylephrine dose-dependently increased protein synthesis by H9C2 cardiomyocytes (EC₅₀ is 5.5·10⁻¹⁰; FIG. 2D), which also associated with increased EGFP fluorescence (R² is 0.5734, p<0.0001; FIG. 2E). The addition of a broadly effective protein synthesis inhibitor Brefeldin A inhibits the phenylephrine-induced increase in EGFP fluorescence (FIG. 2F), suggesting that EGFP is newly synthesized upon phenylephrine stimulation. Thereby, the experimental set-up is validated.

Sul-Compounds Inhibit H9C2 Cardiomyocyte Hypertrophic Responses

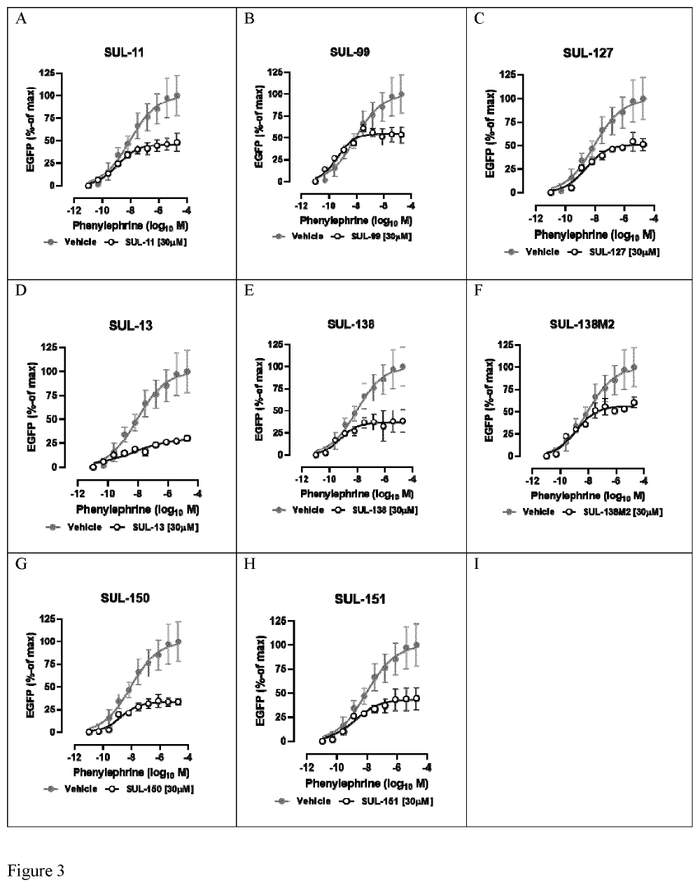

Phenylephrine dose-dependently induces the expression of EGFP in H9C2 cardiomyocytes (FIGS. 3A-H, grey lines). Pre-incubation of H9C2 cardiomyocytes with 30 μM of either SUL-11, -99, -127, -13, -138, -138M2, -150 or -151 reduced the expression of EGFP induced by phenylephrine. However, compounds according the invention (SUL-13, -138, -150 or -151) clearly showed increased potencies and efficacies (Table 2). Of note, SUL-138M2, the primary metabolite of SUL-138, has efficacy to inhibit EGFP expression compared to SUL-138, suggesting that SUL-138 may act as pro-drug in this experiment. Furthermore, the R- and S-enantiomers of (6-hydroxy-2,5,7,8-tetramethylchroman-2-yl)(piperazin-1-yl)methanone, SUL 150 and SUL-151 respectively, were of comparable efficacy in inhibiting EGFP expression (FIG. 2 G and H, respectively) by H9C2 cardiomyocytes.

The results are furthermore summarized in Table 3

TABLE 3

| | Phenylephrine | | SUL |
|---|---|---|---|
| | EC₅₀ (M) | Eₘₐₓ (%) | EGFP |
| Compound | (95% CI) | (95% CI) | Inhibition (%) |
| Vehicle | 9.01 · 10⁻⁹ | 100 | 0 |
| reference | (1.36 · 10⁻⁹-2.97 · 10⁻⁸) | (80.09-100) | |
| SUL-11 | 1.22 · 10⁻⁹ | 45.95 | 54.05 |
| reference | (4.07 · 10⁻¹⁰-4.23 · 10⁻⁹) | (40.75-53.50) | |
| SUL-99 | 4.06 · 10⁻¹⁰ | 54.89 | 55.11 |
| reference | (1.35 · 10⁻¹⁰-1.10 · 10⁻⁹) | (49.51-60.88) | |
| SUL-127 | 2.41 · 10⁻⁹ | 51.22 | 48.78 |
| reference | (8.85 · 10⁻¹⁰-8.27 · 10⁻⁹) | (45.29-59.66) | |
| SUL-13 | 4.76 · 10⁻⁹ | 31.15 | 68.85 |
| invention | (6.64 · 10⁻¹⁰-1.56 · 10⁻⁶) | (25.47-52.57) | |
| SUL-138 | 6.29 · 10⁻¹⁰ | 36.82 | 63.18 |
| invention | (6.29 · 10⁻¹¹-2.15 · 10⁻⁸) | (29.17-54.29) | |
| SUL-138M2 | 1.26 · 10⁻⁹ | 56.08 | 43.92 |
| invention | (4.58 · 10⁻¹⁰-3.59 · 10⁻⁹) | (50.35-63.56) | |
| SUL-150 | 1.81 · 10⁻⁹ | 33.51 | 66.49 |
| invention | (6.19 · 10⁻¹⁰-6.04 · 10⁻⁹) | (29.43-38.90) | |
| SUL-151 | 1.55 · 10⁻⁹ | 43.20 | 56.8 |
| invention | (2.56 · 10⁻¹⁰-4.59 · 10⁻⁸) | (35.29-62.53) | |

### Example 3

The following experiments were designed after finding the unexpected results of example 1. The following findings may be useful to understand why the claimed SUL-type compounds are efficacious in treating heart failure with reduced ejection fraction.

In this example 3, it is shown that DOX-induced heart failure coincides with cardiac fibrogenesis (increased collagen deposition) and oxidative stress (increased lipid peroxidation), that may derive from mitochondrial dysfunction (decreased cardiac mtDNA copy numbers and ATP production).

SUL-150, either administered in a prophylactic or therapeutic model, mitigated these pathological processes and maintained cardiac mitochondrial function at homeostatic levels. In isolated mitochondria, SUL-150 mitigates the DOX-induced reduction of respiratory complex IV activity, which is suggested to be the underlying mechanism for the observed therapeutic effects.

### Experimental Design

Preparation of SUL-150 Food Pellets

SUL-150 (250 mg) was dissolved in 100% EtOH (1.02 ml) and further diluted in distilled water (68.7 ml) to obtain a clear 10 mM solution. The SUL-150 solution was sprayed homogeneously over standard food pellets (1.25 kg). Food pellets were air-dried overnight. Food pellets were freshly prepared weekly. With the approximation of 200 mg·kg⁻¹ SUL-150 in the food pellets and a daily dietary intake of 25 gram for a 350 gram rat, daily dosing of SUL-150 approximates 5 mg·day⁻¹.

Animal Procedures

Male outbred Wistar rats of 250-300 grams (10-12 weeks old) were kept on a 12 h light/dark cycle and received a standard diet with distilled water ad libitum. Thirty rats were administered with doxorubicin (2 mg·kg⁻¹) weekly via intraperitoneal injection for 9 consecutive weeks and were left either untreated (standard chow diet, 10 rats), or received a diet supplemented with SUL-150 as prophylaxis (prior to the 1^{st} doxorubicin administration, 10 rats) or therapy (starting after the 6th doxorubicin administration, 10 rats). Sham control rats (8 rats) were administered with saline weekly via intraperitoneal injection for 9 consecutive weeks and received a standard diet. All rats were subjected to cardiac function assessment 12 weeks after the first doxorubicin administration.

### Experimental Protocols

For the results of the cardiac function tests (PV-Loop assessment) reference is made to example 1 described above.

### Cardiac Fibrosis

Cardiac tissue samples for histopathology were fixed in 3.6% formalin and paraffin-embedded. 4 μm-thick cardiac tissue sections were made and stained using picrosirius red and counterstained with Weighert's hematoxylin (both Sigma-Aldrich, St. Louis, MO) following manufacturer's instruction. Samples were imaged on a NanoZoomer S60 Digital slide scanner (Hammamatsu Photonics) and left-ventricular interstitial fibrosis (i.e. non-perivascular fibrosis) was quantified using Aperio ImageScope (Leica Biosystems, Nussloch, Germany).

### Cardiac Oxidative Stress

Cardiac tissue samples were homogenized in ddH₂O using a TissueRuptor II (Qiagen, Hilden, Germany) followed by sonication at 20 kHz for 3×1 min (Sonopuls 2000, Bandelin, Berlin, Germany) and centrifugation at 14000 g to pellet insoluble proteins. Supernatant was 5 used to assess radical scavenging activity by ABTS-radical decolorization as described by Re et al. [14], lipid peroxidation by assessing the reactivity to thiobarbituric acid according to Ohkawa et al. [15].

### Cardiac Mitochondrial Copy Number

Cardiac tissue samples were homogenized in lysis buffer (100 mM NaCl, 10 mM EDTA, 0.5% SDS in 20 mM Tris-HCl, pH 7.4) containing 50 U·ml⁻¹ RNase I and 100 U·ml⁻¹ proteinase K (both ThermoFisher, Walthham, MA). After overnight incubation at 55° C., total DNA was precipitated using 2-propanol. Aliquots of 5 ng total DNA were amplified on a ViiA7 Real-time PCR system (ThermoFisher, Waltham, MA) using iTaq Universal SYBR Green Supermix (Bio-Rad, Hercules, CA) and primers specific for mitochondrial DNA (MT-ND1; sense 5′-CCTCCTAATAAGCGGCTCCT-3′, antisense 5′-GGCGGGGATTAATAGTCAGA-3′) or nuclear DNA (NDUFA1; sense 5′-ATGGCCCGAACCAAGCAGACC-3′, antisense 5′-TTAAGCTCTCTCCCCCCGTATCCG-3′). MtDNA copy number was calculated as:

mtDNA=2×2^{C1(NDUFA1)-C1(MT-ND1)}.

### Cardiac ATP/ADP Ratio

Cardiac tissue samples were homogenized in Tris-saturated phenol (pH 7.4) using a TissueRuptor II (Qiagen, Hilden, Germany) to extract adenosines and separated over chloroform:water (1:1 v/v) by centrifugation. Nucleotide separation and measurements of ATP and ADP concentration were performed by HPLC. The separation was performed by injection of 100 μL samples into a reversed-phase chromatography C18 column. The column temperature was maintained at 25° C. The mobile phase was 70% acetonitrile:30% 75 mmol L-1 KH2PO4 (v/v) and the flow rate was 1 mL/min. The eluted nucleotides were detected at a wavelength of 260 nm. Nucleotide concentration of the eluate was calculated using calibration curves for each standard nucleotide peak area. ATP concentration was normalized against the concentration of ADP in each sample.

### Respiratory Complex IV Activity

H9C2 cardiomyoblasts were exposed to 1 μM DOX in the presence or absence of 1 μM SUL-150 under standard cell culture conditions for 24 h. Mitochondria were isolated using density gradient centrifugation using the MitoCheck® Mitochondrial Isolation Kit (Cayman Chemical #701010, Ann Arbor, MI) according to manufacturer's instructions and assessed for the activity of mitochondrial complex IV (cytochrome c oxidase) by measuring the rate of oxidation of cytochrome c, which is reflected by an increase in absorbance at 550 nm (Cayman Chemical #700990, Ann Arbor, MI).

### Statistical Evaluation

Data was analysed in GraphPad Prism 8.0 (GraphPad Software Inc, Ca). All results are expressed as mean±S.D. or median and interquartile interval. The difference between groups was assessed by ANOVA followed by pairwise comparisons to the sham and vehicle treatment groups with p-values adjusted for multiple comparisons using FDR correction.

### RESULTS

SUL-150 Administration Appears to Reduce Cardiac Fibrosis in the Rat Model of Doxorubicin-Induced Heart Failure

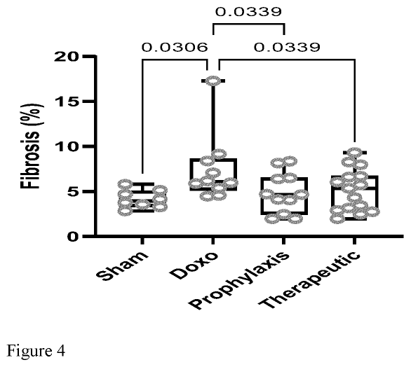

Chronic doxorubicin administration in rats results in the development of heart failure as indicated by a reduction in heart rate, ejection fraction, and stroke work, culminating in a severe reduction in cardiac output. In addition, chronic doxorubicin administration provokes a fibrogenic response in the left ventricle interstitium as indicated by increased collagen deposition (FIG. 4). The administration of SUL-150 via food pellets, either prior to the administration of doxorubicin (prophylaxis group), or after 6 cumulative doxorubicin administrations (therapeutic group) maintains cardiac function (as shown above) and blocks this fibrogenic reaction (FIG. 4).

SUL-150 Administration Appears to Mitigate Cardiac Oxidative Stress Without Affecting Cardiac Antioxidant Capacity

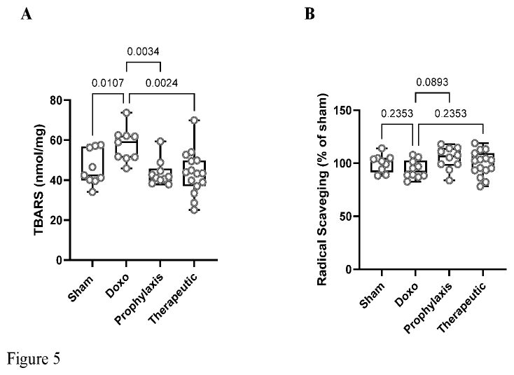

Heart failure is associated with increased oxidative stress in the cardiac tissue, which may result from an imbalance in oxidative radical scavenging capacity and oxidative radical production. Cardiac oxidative stress was evident following chronic doxorubicin administration, as indicated by increased lipid peroxidation products (TBARS, FIG. 5A). The administration of SUL-150, either in prophylaxis or therapeutic regime, mitigates cardiac lipid peroxidation (FIG. 5A) suggestive of a reduction in cardiac oxidative stress.

Cardiac radical scavenging activity remained unaltered by chronic doxorubicin administration (FIG. 5B), nor by the prophylactic or therapeutic administration of SUL-150 (FIG. 5B), suggesting that increased radical production rather than decreased scavenging capacity underlies the observed increase in oxidative stress.

SUL-150 Administration Maintains Cardiac Mitochondrial Copy Number and Normalizes Cardiac Energy Status

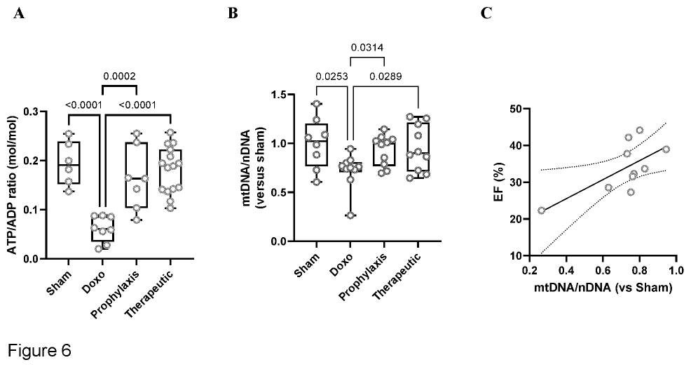

Heart failure is associated with cardiac energy loss, the development of mitochondrial dysfunction and a loss of mitochondrial mass through mitophagy, resulting in contractile dysfunction. Indeed, chronic doxorubicin administration reduced the cardiac ATP content available for contraction (FIG. 6A), which was mitigated by the prophylactic or therapeutic administration of SUL-150.

Mitochondrial DNA (mtDNA) copy numbers can be used as a surrogate marker for mitochondrial mass, and a reduction in mitochondrial mass may underly the energy depletion that occurs after chronic doxorubicin administration. Chronic doxorubicin administration reduced the cardiac mtDNA copy number (FIG. 6B), which is precluded by the administration of SUL-150. Of note, in doxorubicin-treated animals, mtDNA copy number positively associates with cardiac ejection fraction (r²=0.454, p=0.033; FIG. 6C), suggesting that a higher mitochondrial load corresponds to a better contractility of the heart.

SUL-150 Mitigates the Doxorubicin-Induced Reduction of Respiratory Complex IV Activity in Isolated Cardiac Mitochondria

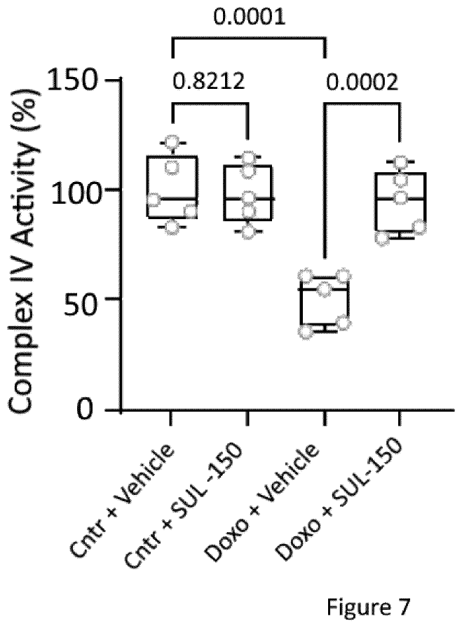

SUL compounds have been shown to increase mitochondrial function through the activation of respiratory complex IV, which may be inhibited by doxorubicin. Indeed, when H9C2 cardiomyoblast cells are exposed to doxorubicin (1 μM for 24 h), the activity of respiratory complex IV is markedly reduced (FIG. 7). Co-incubation of H9C2 cardiomyoblasts with doxorubicin and SUL-150 (both 1 μM for 24 h) maintains complex IV activity, which may explain the enhanced ATP production observed in SUL-150 treated rats. Interestingly, in untreated control H9C2 cardiomyoblasts, the administration of SUL-150 (1 μM for 24 h) does not alter the activity of respiratory complex IV.

### CONCLUSION

The chemotherapeutic agent Doxorubicin is known for its cardiotoxic effects, culminating in heart failure with reduced ejection fraction (HFrEF).

Example 1 shows that the 6-chromanol SUL-150 (administered either prophylactic or therapeutic) maintains cardiac contractile function and thus cardiac output despite administration of doxorubicin.

Example 2 shows that analogues of SUL-150 as claimed also are efficacious in preventing ventricular hypertrophy; and notably are more efficacious than other Trolox-type compounds that are not according the invention, which shows that these claimed compounds can also be used in the treatment of HFrEF

Example 3 shows that doxorubicin-induced heart failure coincides with an increase in cardiac oxidative stress, energy depletion and a loss of mitochondrial mass. SUL-150 administration, either prophylactically or therapeutically, unexpectedly precludes these pathological changes, potentially through maintenance of respiratory complex IV activity.

1. Compound according to formula (I) or (II), the hydroquinone analogue of formula (II), or a pharmaceutically acceptable salt thereof for use in the treatment or prophylaxis of heart failure with a reduced ejection fraction; 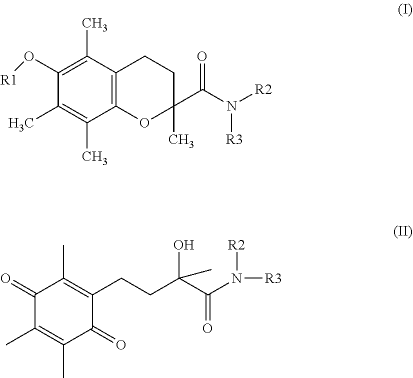
wherein R1 represents a hydrogen or prodrug moiety that can be removed in living tissue
and wherein either
R2 and R3 together with the N atom to which they are attached form a saturated or unsaturated, non-aromatic, optionally substituted, 5-8 membered ring, having one to four N, O, or S atoms, wherein R2 and R3 together contain 3-12 carbon atoms;
or R2 is a hydrogen atom, or an alkyl group with 1-6 carbon atoms, and R3 is an alkyl group, optionally substituted with nitrogen or oxygen, wherein the alkyl group comprises 3-12 carbon atoms, and wherein the alkyl group in R3 comprises one or more non-aromatic cyclic structures that may comprise nitrogen or oxygen atoms in the ring, and may contain linear and/or branched substituted groups, and one or more ethylenic unsaturations.

2. Compound for use according to claim 1, wherein R1 is hydrogen or forms together with the 6-oxygen an ester group with 2-6 carbon atoms.

3. Compound for use according to any one of claims 1-2, wherein the nitrogen in R2 and/or R3 can be amine, quaternary amine, guanidine, or imine and the oxygen in R2 and/or R3 can be hydroxyl, carbonyl or carboxylic acid; and/or oxygen and nitrogen in R2 and/or R3 together can form amide, urea or carbamate groups.

4. Compound for use according to any one of claims 1-3, wherein in either compounds according to formula (I) or according to formula (II), R2 and R3 together with the N atom to which they are attached form a saturated ring incorporating an additional N atom, which ring is unsubstituted or substituted with an alcohol, or alkanol group having 1-4 carbon atoms.

5. Compound for use according to any one of claims 1-4, wherein the compound is a compound according to formula I.

6. Compound for use according to claim 5, wherein R2 and R3 together with the N atom to which they are attached form a 5-7 membered ring comprising one additional amine group, which ring is optionally substituted with methyl, ethyl, or alcohol substituted methyl or ethyl.

7. Compound for use according to any one of claims 1-3, wherein R2 is a hydrogen atom and R3 comprises a saturated cyclic structure having 4-7 carbon atoms and having one nitrogen atom, which ring may be substituted with an alkyl group, alcohol group, or with a group with 1-4 carbon atoms that may comprise an oxygen, carboxylic acid or amine group.

8. Compound for use according to claim 7, wherein the compound is a compound according to formula II and wherein R2 is a hydrogen atom and R3 comprises a cyclic structure having 4-6 carbon atoms and having one nitrogen atom which ring is optionally substituted with methyl, ethyl, or alcohol substituted methyl or ethyl.

9. Compound for use according to claim 1, wherein the compound is (6-hydroxy-2,5,7,8-tetramethylchroman-2-yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13) or (6-hydroxy-2,5,7,8-tetramethylchroman-2-yl)(4-(2-hydroxyethyl)piperazin-1-yl)methanone (SUL-109) or a pharmaceutically acceptable salt thereof, as a racemic mixture or as one of its enantiomers.

10. Compound for use according to claim 9, wherein the compound is the 2R-enantiomer of SUL-121: (2R)-(6-hydroxy-2,5,7,8-tetramethylchroman-2yl)(piperazin-1-yl)methanone (SUL-150) or a pharmaceutically acceptable salt thereof.

11. Compound for use according to any of claims 1-8, wherein the compound according formula (I) or formula (II) has a molecular weight lower than 500 Da.

12. Compound for use according to any of the preceding claims, wherein the treatment or prophylaxis is done in a combination therapy with one or more common measures to treat heart failure.

13. Compound for use according to any of the preceding claims wherein the patient having heart failure with a reduced ejection fraction has a preserved ejection fraction of 50% or lower (HFmrEF)

14. Compound for use according to claims 13, wherein the patient having heart failure with a reduced ejection fraction has a preserved ejection fraction and of about 40% or lower (HFrEF).
